SEQUENCE LISTING (14 sequences)

SEQ ID NO: 1 - DNA, 25 bp, Artificial Sequence
  Forward primer for PCR amplification of bacterial 16S rRNA gene segment
attagataccctggtagtccacgcc

SEQ ID NO: 2 - DNA, 20 bp, Artificial Sequence
  Reverse primer for PCR amplification of bacterial 16S rRNA gene segment
cgtcatccccaccttcctcc

SEQ ID NO: 3 - DNA, 25 bp, Artificial Sequence
  Forward primer for PCR amplification of Staphylococcus 16S rRNA gene segment
cctataagactgggataacttcggg

SEQ ID NO: 4 - DNA, 24 bp, Artificial Sequence
  Reverse primer for PCR amplification of Staphylococcus 16S rRNA gene segment
ctttgagtttcaaccttgcggtcg

SEQ ID NO: 5 - DNA, 20 bp, Artificial Sequence
  Forward primer for PCR amplification of CoNS coa gene segment
agtggcacagccattagtta

SEQ ID NO: 6 - DNA, 20 bp, Artificial Sequence
  Reverse primer for PCR amplification of CoNS coa gene segment
tgtttcagatgcttgaggtt

SEQ ID NO: 7 - DNA, 23 bp, Artificial Sequence
  Forward primer for PCR amplification of MRSA mecA gene segment
tccaggaatgcagaaagaccaag

SEQ ID NO: 8 - DNA, 25 bp, Artificial Sequence
  Reverse primer for PCR amplification of MRSA mecA gene segment
gacacgatagccatcttcatgttgg

SEQ ID NO: 9 - DNA, 18 bp, Artificial Sequence
  Forward primer for PCR amplification of Pseudomonas oprL gene segment
gtgggttgctcctccaag

SEQ ID NO: 10 - DNA, 20 bp, Artificial Sequence
  Reverse primer for PCR amplification of Pseudomonas oprL gene segment
ctcgaagtagaaggtggtga

SEQ ID NO: 11 - DNA, 20 bp, Artificial Sequence
  Forward primer for PCR amplification of E. Coli lamB gene segment
agcgcagatggaaatcaacc

SEQ ID NO: 12 - DNA, 19 bp, Artificial Sequence
  Reverse primer for PCR amplification of E. Coli lamB gene segment
caaatgcaacgccagaacc

SEQ ID NO: 13 - DNA, 22 bp, Artificial Sequence
  Forward primer for PCR amplification of Enterococcus ent gene segment
tactgacaaaccattcatgatg

SEQ ID NO: 14 - DNA, 21 bp, Artificial Sequence
  Reverse primer for PCR amplification of Enterococcus ent gene segment
aacttcgtcaccaacgcgaac

(19) United States
(12) Patent Application Publication
(10) Pub. No.: US 2015/0376684 A1
(43) Pub. Date: Dec. 31, 2015

(54) TEST KIT AND METHOD FOR RAPIDLY DETECTING LIVE BACTERIUM AND TEST KIT AND METHOD FOR RAPIDLY DETERMINING APPROPRIATE ANTIBIOTIC

(71) Applicant: NATIONAL TSING HUA UNIVERSITY, HSINCHU (TW)

(72) Inventors: Gwo-Bin LEE, HSINCHU (TW); Shiuann-Sheng LEE, TAIPEI (TW); Chih-Hung WANG, HSINCHU (TW); Wen-Hsin CHANG, HSINCHU (TW)

(21) Appl. No.: 14/507,842

(22) Filed: Oct. 7, 2014

(30) Foreign Application Priority Data
Jun. 25, 2014 (TW) 103121935

(51) Int. Cl. C12Q 1/68

(52) U.S. Cl. CPC C12Q 1/689; C12Q 2600/158

(57) ABSTRACT

A test kit for rapidly detecting a live bacterium is disclosed. The test kit for rapidly detecting a live bacterium includes ethidium monoazide, a magnetic bead, a first primer pair and a microfluidic chip. A test kit for rapidly determining an appropriate antibiotic is also disclosed. The test kit for rapidly determining an appropriate antibiotic includes ethidium monoazide, a magnetic bead, four primer pairs and a microfluidic chip.

### RELATED APPLICATIONS

This application claims priority to Taiwan Application Serial Number 103121935, filed Jun. 25, 2014, which is herein incorporated by reference.

### BACKGROUND

1. Technical Field

The present disclosure relates to a test kit and method for rapidly detecting a bacterium. More particularly, the present disclosure relates to a test kit and method for rapidly detecting a live bacterium and a test kit and method for rapidly determining an appropriate antibiotic.

2. Description of Related Art

At present, the microbiological culture, for a period of five to seven days, is the conventional practice for identifying an infecting bacterium of a sample in hospital. The result of the microbiological culture is accurate; however, the process is time-consuming. The patient has been treated with broad-spectrum antibiotics before the infecting bacterial strain is identified. The broad-spectrum antibiotics could easily lead to the generation of resistant bacteria, causing difficulties in subsequent treatment.

Although there are some methods to rapidly detect the existence of bacteria in hospital, such as by measuring the level of C-reactive protein or interleukin-6 in serum, these methods are non-specific. Therefore, clinically, the antibiotic selection is relied on the speculation of physicians, and then adjusted in conjunction with observed responses of the patient who has been treated with broad-spectrum antibiotics. Finally, the really appropriate antibiotic selection is made based on the susceptibility test report for the bacterium. As such, it not only takes much time, but also may cause deteriorated condition of the patient due to the inappropriate choice of antibiotics, not to mention results in the public health crisis because of the transfer of drug-resistant genes due to undue use of antibiotics. Moreover, nowadays many patients may have been treated with different doses of antibiotics before receiving the standard therapy because of the overuse of antibiotics, so that the microbiological culture assay may present a false-negative result since the activity of bacteria is reduced by the antibiotics.

Molecular diagnosis such as polymerase chain reaction (PCR) has been applied to quickly detect the existence of bacteria in a sample in a short time. However, in the PCR, DNA or RNA is used as template, and residual DNA of dead bacteria is also present in the sample, which may cause a false-positive result. Using RNA as template can avoid this problem, but unfortunately, a purified specimen must be used in PCR and the extraction of RNA is comparatively complicated and RNA is readily degraded, which increases the complexity and difficulty of the detection.

### SUMMARY

In one aspect, a test kit for rapidly detecting a live bacterium is provided. The test kit includes ethidium monoazide, a magnetic bead, a first primer pair and a microfluidic chip. The ethidium monoazide can intercalate into a DNA of a dead bacterium. The magnetic bead is bonded with vancomycin for capturing Gram-positive and Gram-negative bacteria. The first primer pair includes the nucleotide sequences referenced as SEQ ID NOs: 1 and 2. The microfluidic chip includes a fluid storage unit, a reaction unit, a fluid transportation unit, and a valve unit. The fluid storage unit includes a first fluid storage chamber for storing a wash buffer and a second fluid storage chamber for storing a PCR reagent. The reaction unit includes a reaction chamber, a positive control reaction chamber and a negative control reaction chamber. The fluid transportation unit includes a pneumatic micro-pump and a transportation unit control air hole, wherein the fluid transportation unit is disposed between the fluid storage unit and the reaction unit for transporting the wash buffer or the PCR reagent to the reaction unit. The valve unit includes a plurality of pneumatic micro-valves and a plurality of valve control air holes. The pneumatic micro-valves are disposed between the fluid storage unit and the pneumatic micro-pump, and between the pneumatic micro-pump and the reaction unit. The valve control air holes supply air into the pneumatic micro-valves for controlling opening or closing of the pneumatic micro-valves.

In another aspect, a test kit for rapidly determining an appropriate antibiotic is provided. The test kit includes ethidium monoazide, a magnetic bead, a plurality of primer pairs and a microfluidic chip. The ethidium monoazide can intercalate into a DNA of a dead bacterium. The magnetic bead is bonded with vancomycin for capturing Gram-positive and Gram-negative bacteria. The primer pairs includes a first primer pair including the nucleotide sequences referenced as SEQ ID NOs: 1 and 2, a second primer pair including the nucleotide sequences referenced as SEQ ID NOs: 3 and 4, a third primer pair including the nucleotide sequences referenced as SEQ ID NOs: 5 and 6, and a fourth primer pair including the nucleotide sequences referenced as SEQ ID NOs: 7 and 8. The microfluidic chip includes a plurality of modules, which can simultaneously perform PCR reactions. Each of the modules includes a fluid storage unit, a reaction unit, a fluid transportation unit and a valve unit. The fluid storage unit includes a first fluid storage chamber for storing a wash buffer and a second fluid storage chamber for storing a PCR reagent. The reaction unit includes a reaction chamber, a positive control reaction chamber and a negative control reaction chamber. The fluid transportation unit includes a pneumatic micro-pump and a transportation unit control air hole, wherein the fluid transportation unit is disposed between the fluid storage unit and the reaction unit for transporting the wash buffer or the PCR reagent to the reaction unit. The valve unit includes a plurality of pneumatic micro-valves and a plurality of valve control air holes. The pneumatic micro-valves are disposed between the fluid storage unit and the pneumatic micro-pump, and between the pneumatic micro-pump and the reaction unit. The valve control air holes supply air into the pneumatic micro-valves for controlling opening or closing of the pneumatic micro-valves.

### BRIEF DESCRIPTION OF THE DRAWINGS

The present disclosure can be more fully understood by reading the following detailed description of embodiments, with reference to the accompanying drawings as follows:

FIG. 1 is a schematic view of a microfluidic chip according to a first embodiment of the present disclosure;

FIG. 2 is a side view of the microfluidic chip of FIG. 1;

FIG. 3 is a schematic side view of fluid flow in the microfluidic chip of FIG. 1;

FIG. 4 shows the results of fluorescent analysis and gel electrophoresis according to the first embodiment of the present disclosure;

FIG. 5 is a schematic view of a microfluidic chip according to a second embodiment of the present disclosure;

FIG. 6 shows the results of fluorescent analysis and gel electrophoresis using a second primer pair according to the second embodiment of the present disclosure;

FIG. 7 shows the results of fluorescent analysis and gel electrophoresis using a third primer pair according to the second embodiment of the present disclosure;

FIG. 8 shows the results of fluorescent analysis and gel electrophoresis using a fourth primer pair according to the second embodiment of the present disclosure;

FIG. 9 shows the results of fluorescent analysis and gel electrophoresis using a fifth primer pair according to the second embodiment of the present disclosure;

FIG. 10 shows the results of fluorescent analysis and gel electrophoresis using a sixth primer pair according to the second embodiment of the present disclosure; and

FIG. 11 shows the results of fluorescent analysis and gel electrophoresis using a seventh primer pair according to the second embodiment of the present disclosure.

### DETAILED DESCRIPTION

A test kit and method for rapidly detecting a live bacterium are provided. A nucleic acid fluorescent stain is used to intercalate into a DNA of a dead bacterium, so that nucleic acid amplification of the dead bacterium by PCR is prevented. Next, a magnetic bead capable of capturing bacteria indiscriminately is used to isolate the bacteria from the sample. Then, an integrated microfluidic chip is used to automatically perform the overall infecting bacterium detection process, including differentiation of live and dead bacteria, purification of pathogenic bacteria, washing out of impurities, PCR, and interpretation of reaction results. The bacterial detection procedure can be done within 1 hour. Accordingly, a test kit and method for rapidly determining an appropriate antibiotic are also provided.

The following examples and embodiments are used to illustrate the present disclosure, which enables one skilled in the art to fully make and use the present disclosure without undue experimentations. These embodiments are only used to explain how to implement the materials and methods of the present disclosure, and should not be interpreted as limiting to the scope of the present disclosure in any manner.

Experiment 1: Preparation of Ethidium Monoazide

Ethidium monoazide, a nucleic acid stain, can selectively penetrate into a dead bacterium with an incomplete cell membrane to intercalate into the DNA of the dead bacterium. The intercalated ethidium monoazide can be activated with high-intensity visible light to covalently bind to the DNA of the dead bacterium. The resulting covalent complex DNA-ethidium monoazide is stable even at 95° C., thereby preventing downstream PCR amplification. On the other hand, a live bacterium has an intact cell membrane, which can prevent penetration of the ethidium monoazide to intercalate into its DNA, such that downstream PCR amplification can be performed. In this way, the live bacterium and the dead bacterium is distinguished from each other.

Ethidium monoazide (MOLECULAR PROBES® (USA)) is dissolved in ethanol to prepare 1 mg/ml of a stock. The stock is diluted in ethanol to a desired concentration before performing the diagnosis assay. Note that the ethidium monoazide stock should be stored at −20° C. and kept from light.

Experiment 2: Preparation of Magnetic Bead

Vancomycin has a cup-like structure, and can specifically bind to peptidoglycan on the cell wall of both Gram-positive and Gram-negative bacteria through five hydrogen bonds. Therefore, the magnetic bead bonded with vancomycin of the present disclosure can be used for capturing both Gram-positive and Gram-negative bacteria.

To prepare the magnetic bead bonded with vancomycin, 950 μl of magnetic bead (4×10⁶ beads/ml), 30 μl of 100 nM vancomycin (Sigma-Aldrich Co. LLC., USA) and 20 μl of 120 mg/ml 1-ethyl-3-(3-dimethylaminopropyl) carbodiimide hydrochloride are mixed at 4° C. overnight. Then, the mixture is washed with 0.02% Tween 20 in 1 ml phosphate buffer saline (PBS) and 0.1% sodium dodecyl sulfate in 1 ml PBS for 3 minutes respectively, and a part of the magnetic bead that is not bonded with vancomycin is blocked with ethanol amine. Then, 1 ml PBS is added to wash out the residual ethanol amine and the magnetic bead is re-dissolved in 1 ml PBS and stored at 4° C. until use.

Experiment 3: PCR Primer Design and PCR Conditions

Primer pairs used in the present disclosure include a first primer pair including the nucleotide sequences referenced as SEQ ID NOs: 1 and 2, a second primer pair including the nucleotide sequences referenced as SEQ ID NOs: 3 and 4, a third primer pair including the nucleotide sequences referenced as SEQ ID NOs: 5 and 6, a fourth primer pair including the nucleotide sequences referenced as SEQ ID NOs: 7 and 8, a fifth primer pair including the nucleotide sequences referenced as SEQ ID NOs: 9 and 10, a sixth primer pair including the nucleotide sequences referenced as SEQ ID NOs: 11 and 12, and a seventh primer pair including the nucleotide sequences referenced as SEQ ID NOs: 13 and 14. The first primer pair targets a conserved region of bacterial 16S rRNA gene for detecting the existence of bacteria. The second primer pair targets the 16S rRNA gene of Staphylococcus for identifying Staphylococcus. The third primer pair targets the coa gene of Staphylococcus for identifying Coagulase-negative staphylococcus (CoNS). If there is no amplified PCR product, it is indicative of the existence of CoNS in the sample. The fourth primer pair targets the mecA gene of Methicillin-resistant Staphylococcus aureus (MRSA) for identifying MRSA. The fifth primer pair targets the oprL gene of Pseudomonas for identifying Pseudomonas. The sixth primer pair targets the lamB gene of Escherichia coli (E. coli) for identifying E. coli. The seventh primer pair targets the ent gene of Enterococcus for identifying Enterococcus.

The PCR conditions are as follows. KAPA SYBR FAST kit (KAPABIOSYSTEM, USA) is used as PCR reagent. The 20-μl mixture for each reaction contains 10 μl of KAPA SYBR FAST PCR master mix, 0.4 μl of 10 μM forward primer, 0.4 μl of 10 μM reverse primer, and 9.2 μl of distilled water. The mixture is homogenized for PCR reaction. The thermal-cycling conditions include denaturation at 95° C. for 5 minutes, followed by 25 cycles of denaturation at 95° C. for 10 seconds, annealing at 62° C. for 20 seconds and extension at 72° C. for 10 seconds.

### Embodiment 1

According to a first embodiment of the present disclosure, a test kit for rapidly detecting a live bacterium is provided. The test kit includes ethidium monoazide, the magnetic bead, the first primer pair and a microfluidic chip 100. In order to achieve automation, the overall detection process, including sample purification, sample mixing, washing out of impurities, PCR, and interpretation of reaction results, is integrated on the microfluidic chip 100.

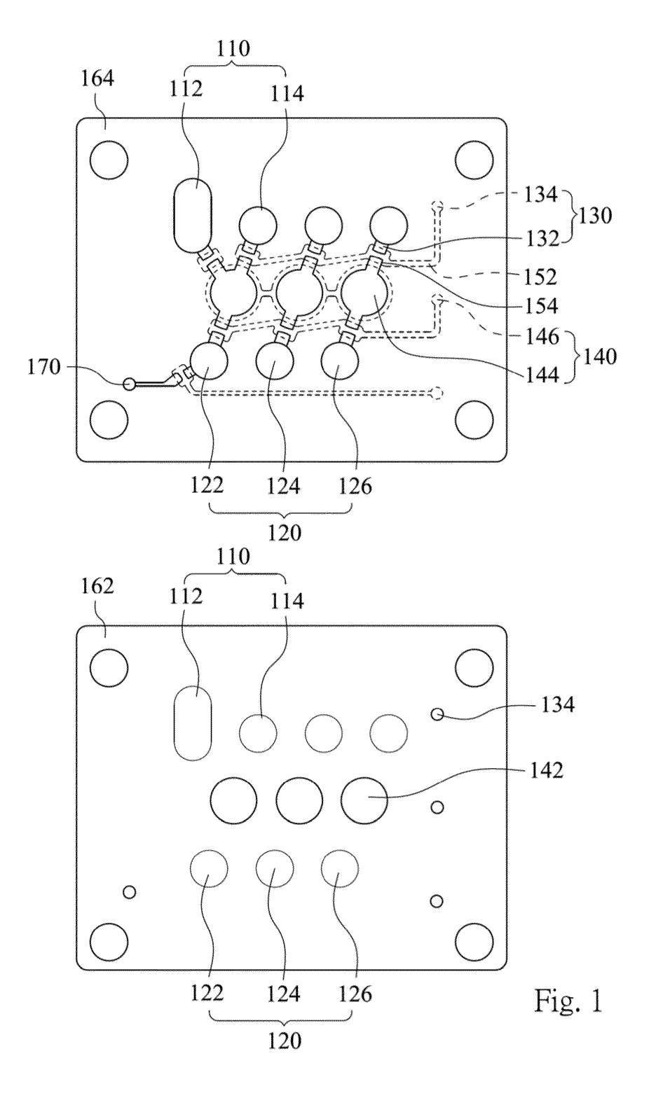

FIG. 1 is a schematic view of the microfluidic chip 100 according to the first embodiment of the present disclosure. In FIG. 1, the microfluidic chip 100 includes a fluid storage unit 110, a reaction unit 120, a fluid transportation unit 140 and a valve unit 130. The fluid storage unit 110 includes one first fluid storage chamber 112 for storing a wash buffer and three second fluid storage chambers 114 for storing a PCR reagent. The reaction unit 120 includes a reaction chamber 122, a positive control reaction chamber 124 and a negative control reaction chamber 126. The fluid transportation unit 140 includes a pneumatic micro-pump 144 and a transportation unit control air hole 146, wherein the fluid transportation unit 140 is disposed between the fluid storage unit 110 and the reaction unit 120 for transporting the wash buffer or the PCR reagent to the reaction unit 120. The valve unit 130 includes a plurality of pneumatic micro-valves 132 and a plurality of valve control air holes 134. The pneumatic micro-valves 132 are disposed between the fluid storage unit 110 and the pneumatic micro-pump 144, and between the pneumatic micro-pump 144 and the reaction unit 120. A flow channel 154 provides for fluid communication between the pneumatic micro-valves 132 and the pneumatic micro-pump 144. The valve control air holes 134 supply air via an airway 152 into the pneumatic micro-valves 132 to open or close the pneumatic micro-valves 132. The microfluidic chip 100 further includes a quantification pillar 142 disposed on the pneumatic micro-pump 144. The quantification pillar 142 can regulate the level of membrane elevation of the pneumatic micro-pump 144 by fluid transportation, resulting in consistent fluid throughout quantification pillar 142.

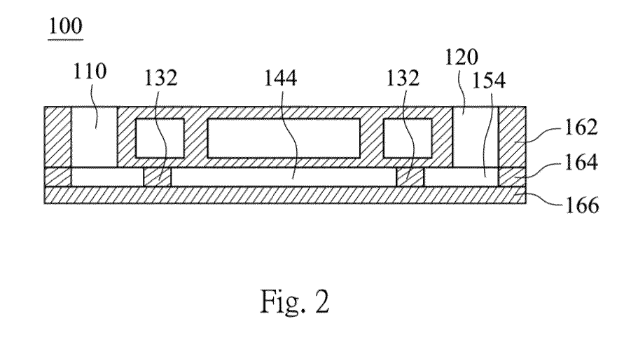

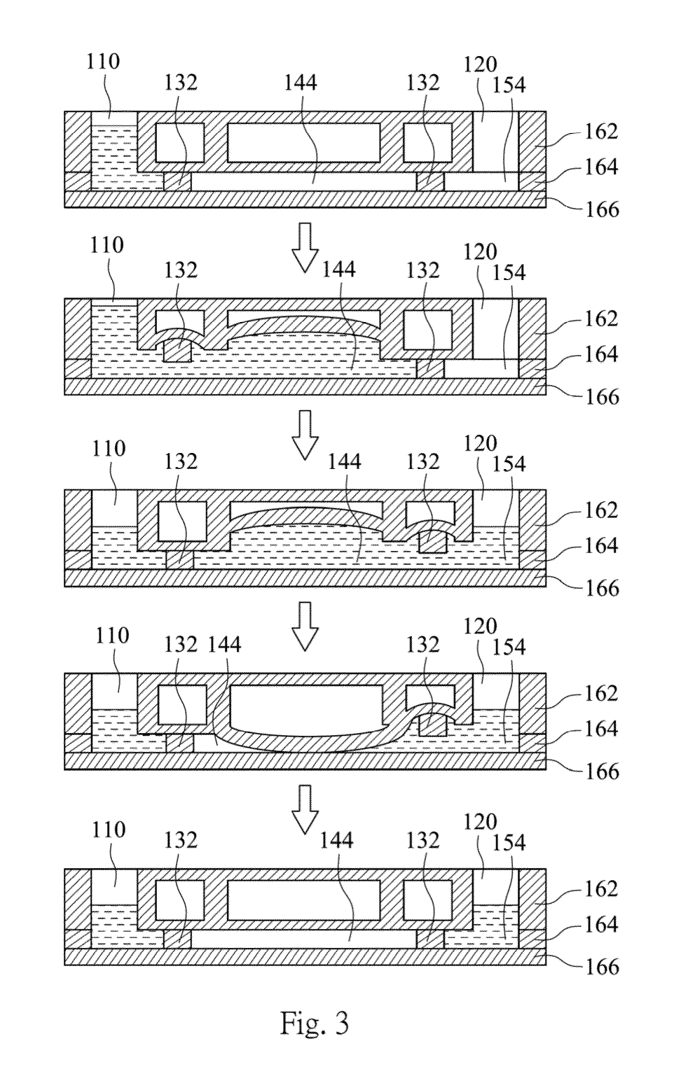

FIG. 2 is a side view of the microfluidic chip 100 of FIG. 1. As shown in FIG. 2, the microfluidic chip 100 is composed of two flexible layers and one glass layer, i.e. a microfluidic chip upper layer 162, a microfluidic chip lower layer 164 and a glass substrate 166 from top to bottom. FIG. 3 is a schematic side view of fluid flow in the microfluidic chip 100 of FIG. 1. As shown in FIG. 3, first, the fluid are loaded in the fluid storage unit 110 and air is supplied by one of the valve control air holes 134 close to the fluid storage unit 110 and the transportation unit control air hole 146. Then, the pneumatic micro-valves 132 and the membrane of the pneumatic micro-pump 144 are elevated by a suction force caused by vacuum, so that the fluid in the fluid storage unit 110 flows into the pneumatic micro-pump 144 via the flow channel 154. Next, air is supplied by one of the valve control air holes 134 close to the reaction unit 120, and another one of the pneumatic micro-valves 132 is elevated, so that the fluid in the pneumatic micro-pump 144 flows into the reaction unit 120 via the flow channel 154. Finally, compressed air is supplied to the pneumatic micro-pump 144 to push all the fluid into the reaction unit 120.

A method for rapidly detecting a live bacterium includes following steps. The washing buffer, the PCR reagent and the first primer pair, and the magnetic bead bonded with vancomycin are pre-loaded into the first fluid storage chamber 112, the second fluid storage chamber 114 and the reaction chamber 122, respectively. Then, a sample mixed with ethidium monoazide is loaded into the reaction chamber 122 and exposed to visible light for 1 to 20 minutes to trigger the formation of the covalent complex DNA-ethidium monoazide reaction, wherein the concentration of ethidium monoazide is 0.01 to 30 mg/mL. The sample, the ethidium monoazide and the magnetic bead bonded with vancomycin are further incubated for 10 min, during which both Gram-positive and Gram-negative bacteria are bound to vancomycin and thus captured by the magnetic bead. Afterwards, a magnet is used to collect the magnetic bead with captured bacteria. The fluid transportation unit 140 is activated to pump the wash buffer into the reaction chamber 122 through the pneumatic micro-valves 132 via the flow channel 154, to wash out unbound materials on the magnetic bead. The waste fluid is sucked away from a waste fluid port 170 after washing the magnetic bead. Further, the fluid transportation unit 140 is activated to pump the PCR reagent mixed with the first primer pair into the reaction chamber 122 through the pneumatic micro-valves 132 via the flow channel 154 for performing PCR. The PCR conditions are as described in experiment 3 and the increase or decrease in temperature of the PCR reaction is regulated by a temperature control module. Finally, the presence of a PCR reaction product is detected by the fluorescence method, wherein the presence of the PCR product is indicative of the existence of a live bacterium in the sample. Note that the overall process of the method for rapidly detecting a live bacterium, including sample purification, sample mixing, washing out of impurities, PCR and interpretation of reaction results, can be done only within 55 minutes by using the test kit of the present disclosure.

The test kit for rapidly detecting a live bacterium of the present disclosure further includes a positive control group and a negative control group for improving the objectivity of the results from the test kit of the present disclosure. The positive control group is a plasmid containing E. coli 16S rRNA gene as amplification template. Following the above detection steps, in the positive control group, the magnetic bead bonded with vancomycin, the ethidium monoazide and the plasmid are loaded in the positive control reaction chamber 124, and in the negative control group, only the magnetic bead bonded with vancomycin and the ethidium monoazide are loaded in the negative control reaction chamber 126. The remaining conditions are as described above, and are not repeated again here.

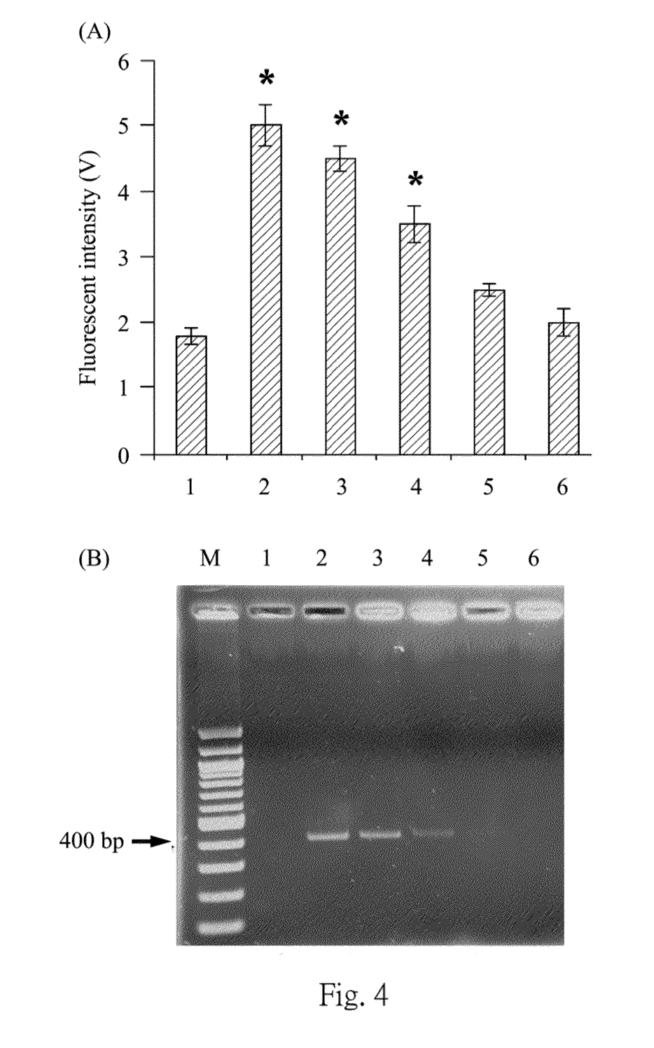

FIG. 4(A) shows the results of fluorescent analysis according to the first embodiment of the present disclosure, and FIG. 4(B) shows the results of gel electrophoresis according to the first embodiment of the present disclosure. Each vertical bars shows mean±S.E. * represents p<0.05 compared with negative control group. M represents the molecular weight of nucleic acid. Lane 1 corresponds to the negative control group. Lanes 2 to 6 correspond to 10⁶ to 10² colony formation unit (CFU)/mL of live E. coli samples. The PCR using the first primer pair can produce an amplification product having an approximately 400 base-pairs (bp) in length, in a sample with live bacteria. The results of either fluorescent analysis or gel electrophoresis indicate that the detection limit of the test kit of the present disclosure is determined to be 10⁴ CFU/mL live bacteria in a sample.

The test kit and method for rapidly detecting a live bacterium of the present disclosure can simplify the detection process and be performed in a fully automatic manner on the microfluidic chip 100. In this way, the problem of false-negative result in the conventional methods is overcome, and the detection results can be rapidly obtained. In addition to reduced reaction time, the amounts of samples and reagents used are reduced, and human-errors are further reduced. The test kit and the method thereof can detect the existence of live bacteria in a sample within 1 hour and have a detection limit of 10 CFU/mL, which is indicative of a high sensitivity. Thus, the test kit and the method thereof can be used on site in a surgical operation, so that information on the existence or absence of a potential pathogen in a patient can be provided in real time, thereby avoiding performing the operation under bacterial infecting conditions.

### Embodiment 2

According to a second embodiment of the present disclosure, a test kit for rapidly determining an appropriate antibiotic is provided. The test kit includes ethidium monoazide, a magnetic bead, four primer pairs and a microfluidic chip 100.

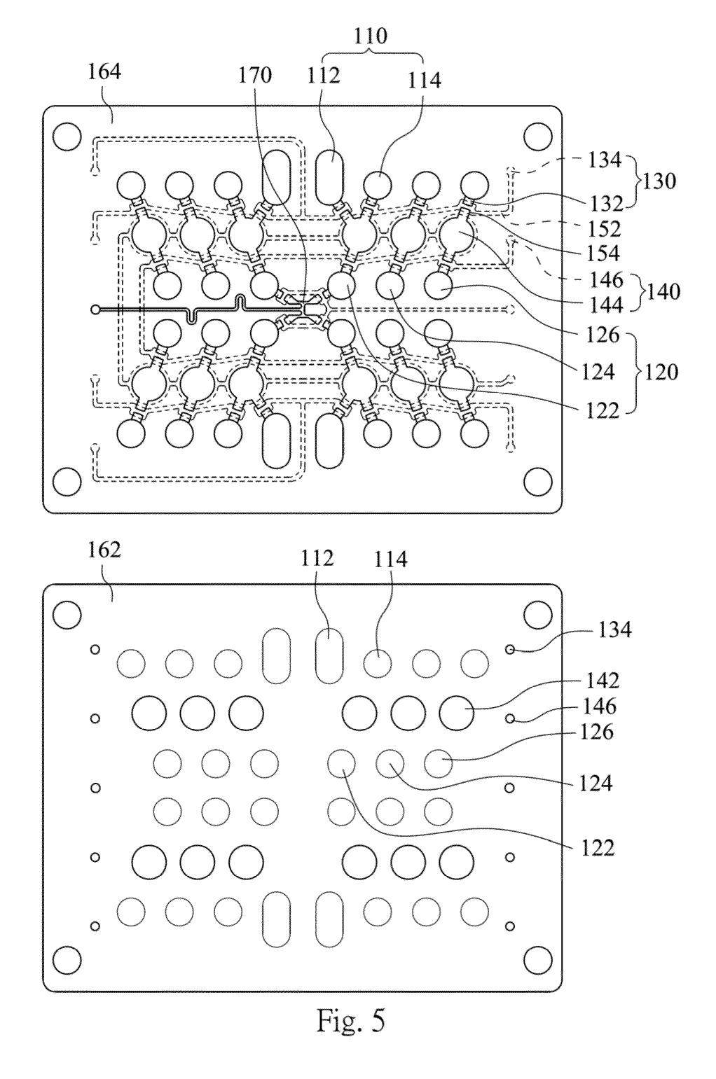

FIG. 5 is a schematic view of the microfluidic chip 100 according to the second embodiment of the present disclosure. In FIG. 5, the microfluidic chip 100 includes four identical modules, which can be used for simultaneously performing four separate tests. Each of the modules includes a fluid storage unit 110, a reaction unit 120, a fluid transportation unit 140 and a valve unit 130. The fluid storage unit 110 includes one first fluid storage chamber 112 for storing a wash buffer and three second fluid storage chambers 114 for storing a PCR reagent. The reaction unit 120 includes a reaction chamber 122, a positive control reaction chamber 124 and a negative control reaction chamber 126. The fluid transportation unit 140 includes a pneumatic micro-pump 144 and a transportation unit control air hole 146, wherein the fluid transportation unit 140 is disposed between the fluid storage unit 110 and the reaction unit 120 for transporting the wash buffer or the PCR reagent to the reaction unit 120. The valve unit 130 includes a plurality of pneumatic micro-valves 132 and a plurality of valve control air holes 134. The pneumatic micro-valves 132 are disposed between the fluid storage unit 110 and the pneumatic micro-pump 144, and between the pneumatic micro-pump 144 and the reaction unit 120. A flow channel 154 provides for fluid communication between the pneumatic micro-valves 132 and the pneumatic micro-pump 144. The valve control air holes 134 supply air via an airway 152 into the pneumatic micro-valves 132 to open or close these valves. The microfluidic chip 100 further includes a quantification pillar 142 disposed on the pneumatic micro-pump 144. The quantification pillar 142 can regulate the level of membrane elevation of the pneumatic micro-pump 144 by fluid transportation, resulting in consistent fluid throughout quantification pillar 142.

A method for rapidly determining an appropriate antibiotic includes following steps. The washing buffer, the PCR reagent and the magnetic bead bonded with vancomycin are pre-loaded into the first fluid storage chamber 112, the second fluid storage chamber 114 and the reaction chamber 122, respectively. Furthermore, four different primer pairs, including a first primer pair, a second primer pair, a third primer pair and a fourth primer pair, are added into the second fluid storage chambers 114 of the four identical modules respectively. Then, a sample mixed with ethidium monoazide is loaded into the reaction chamber 122 and exposed to visible light for 1 to 20 minutes to trigger the formation of the covalent complex DNA-ethidium monoazide, wherein the concentration of ethidium monoazide is 0.01 to 30 mg/mL. The sample, the ethidium monoazide and the magnetic bead bonded with vancomycin are further incubated for 10 min, during which both Gram-positive and Gram-negative bacteria are bound to vancomycin and thus captured by the magnetic bead. Afterwards, a magnet is used to collect the magnetic bead with captured bacteria. The fluid transportation unit 140 is activated to pump the wash buffer into the reaction chamber 122 through the pneumatic micro-valves 132 via the flow channel 154, to wash out unbound materials on the magnetic bead. The waste fluid is sucked away from a waste fluid port 170 after washing the magnetic bead. Further, the fluid transportation unit 140 is activated to pump the PCR reagent mixed with the first primer pair into the reaction chamber 122 through the pneumatic micro-valves 132 via the flow channel 154 for performing PCR. The PCR conditions are as described in experiment 3 and the increase or decrease in temperature of the PCR reaction is regulated by a temperature control module. Finally, the presence of a PCR reaction product is detected by the fluorescence method, and the selection of antibiotic species is determined depending on the PCR amplification product. Note that the PCR product using the first primer pair corresponds to the existence of live bacteria in the sample. The PCR product using the second primer pair corresponds to the existence of live Staphylococcus bacteria in the sample. If there is no PCR product using the third primer pair and there is a PCR product using the second PCR primer pair, it is indicated that live CoNS bacteria are existence in the sample. The PCR product using the fourth primer pair corresponds to the existence of live MRSA bacteria in the sample.

The test kit for rapidly determining an appropriate antibiotic of the present disclosure further includes a fifth primer pair, a sixth primer pair and a seventh primer pair, instead of the second primer pair, the third primer pair and forth primer pair to perform other PCR. Thus, in the detection, the four identical modules of the microfluidic chip 100 include the first primer pair, the fifth primer pair, the sixth primer pair and the seventh primer pair, respectively. Note that the PCR product using the fifth primer pair corresponds to the existence of live Pseudomonas bacteria in the sample. The PCR product using the sixth primer pair corresponds to the existence of live E. coli bacteria in the sample. The PCR product using the seventh primer pair corresponds to the existence of live Enterococcus bacteria in the sample. The detection results by using the second primer pair to the seventh primer pair in the present disclosure can cover 80% pathogenic species in the clinic. Therefore, the test kit of the present disclosure can achieve correct antibiotic use in the first-line treatment, thereby avoiding delay in treatment or generation of resistant bacteria caused by wrong medication.

Furthermore, the test kit for rapidly determining an appropriate antibiotic of the present disclosure further includes a positive control group and a negative control group for improving the objectivity of the results from the test kit of the present disclosure. The positive control group is a plasmid containing E. coli 16S rRNA gene as amplification template. Following the above determination steps, in the positive control group, the magnetic bead bonded with vancomycin, the ethidium monoazide and the plasmid are loaded in the positive control reaction chamber 124, and in the negative control group, only the magnetic bead bonded with vancomycin and the ethidium monoazide are loaded in the negative control reaction chamber 126. The remaining conditions are as described above, and are not repeated again here.

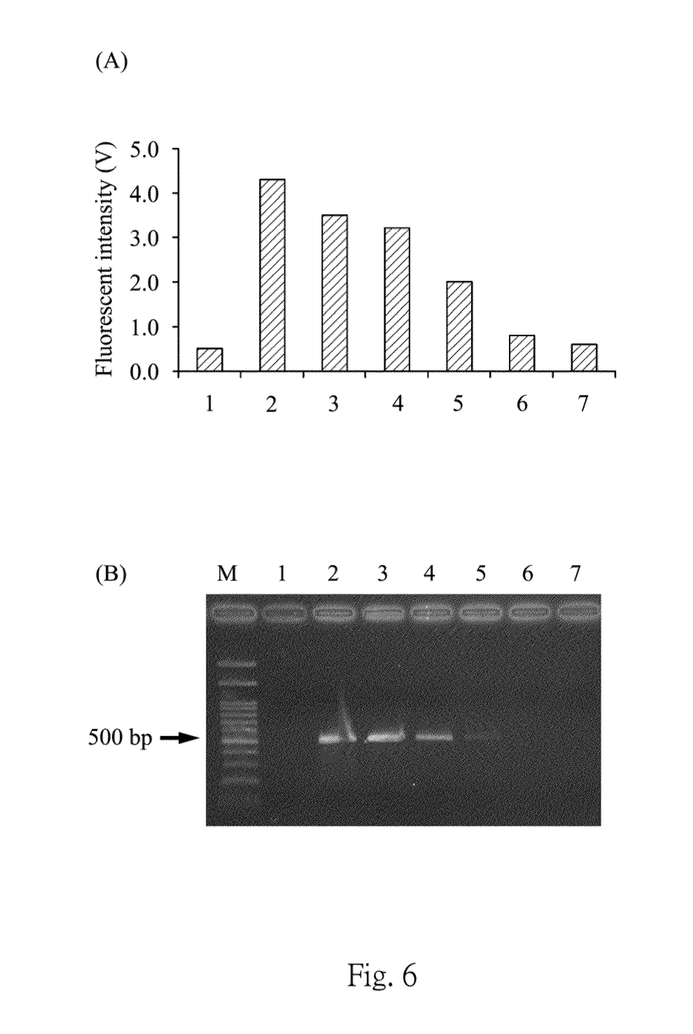

FIG. 6 to FIG. 11 shows the results of fluorescent analysis and gel electrophoresis according to the second embodiment of the present disclosure. M represents the molecular weight of nucleic acid.

FIG. 6(A) represents the results of fluorescent analysis using the second primer pair, and FIG. 6(B) represents the results of gel electrophoresis using the second primer pair. Lane 1 corresponds to the negative control group. Lanes 2 to 7 correspond to 10⁶ to 10¹ CFU/mL of live Staphylococcus samples. The PCR using the second primer pair can produce an amplification product having an approximately 500 base-pairs (bp) in length, in a sample with live Staphylococcus bacteria. The results of either fluorescent analysis or gel electrophoresis indicate that the detection limit of the test kit of the present disclosure is determined to be 10³ CFU/mL live Staphylococcus bacteria in a sample.

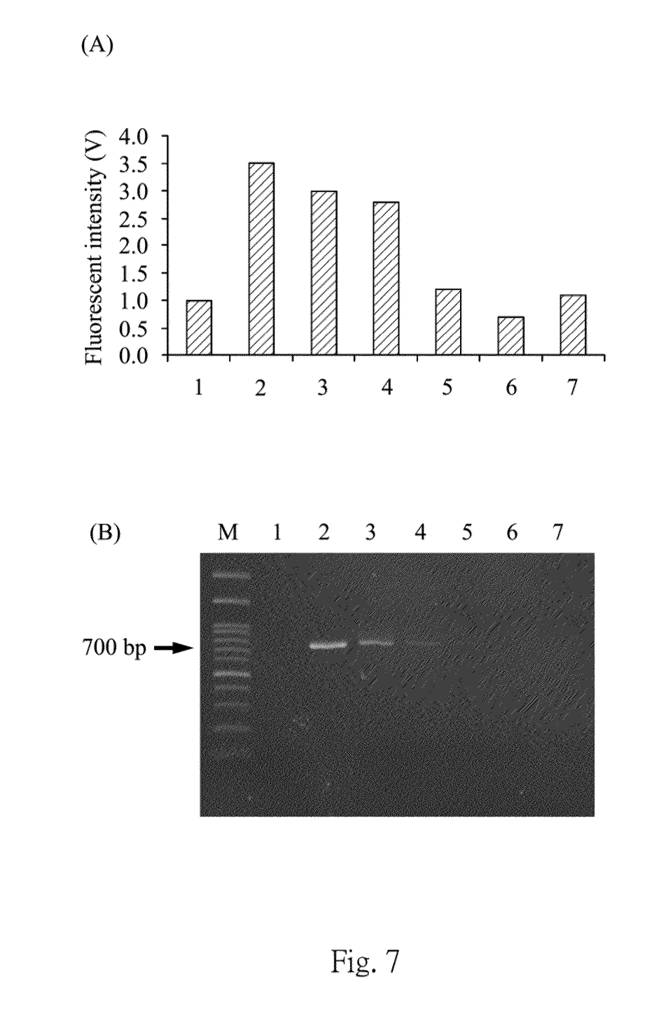

FIG. 7(A) represents the results of fluorescent analysis using the third primer pair, and FIG. 7(B) represents the results of gel electrophoresis using the third primer pair. Lane 1 corresponds to the negative control group. Lanes 2 to 7 correspond to 10⁶ to 10¹ CFU/mL of live Staphylococcus samples. The PCR using the third primer pair can produce an amplification product having an approximately 700 base-pairs (bp) in length, in the sample with live Staphylococcus bacteria. If there is no PCR product using the third primer pair and there is a PCR product using the second PCR primer pair, it is indicative of the existence of CoNS in the sample. The results of either fluorescent analysis or gel electrophoresis indicate that the detection limit of the test kit of the present disclosure is determined to be 10⁴ CFU/mL CoNS bacteria in the sample.

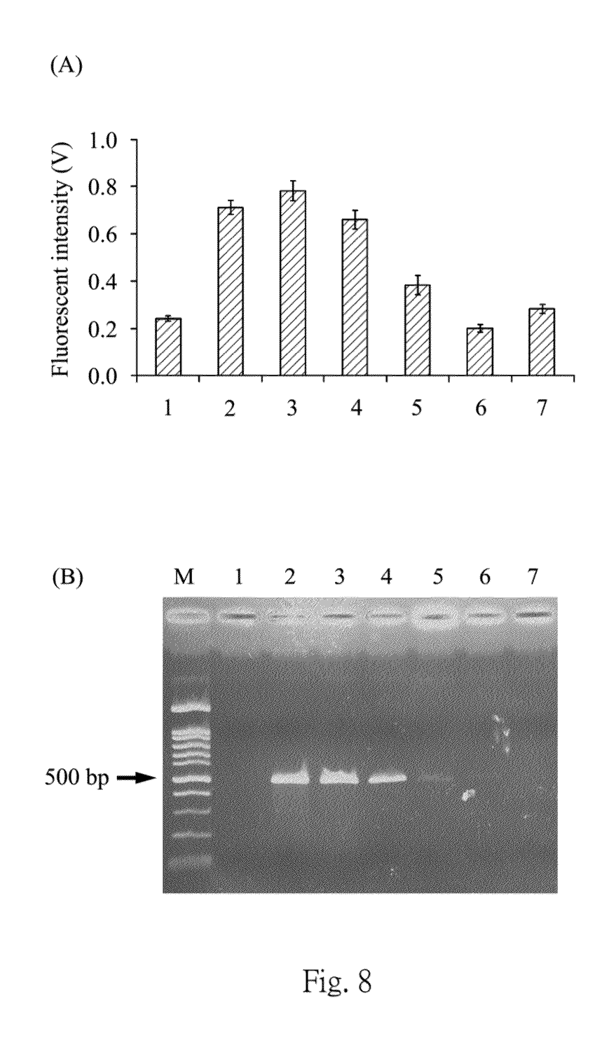

FIG. 8(A) represents the results of fluorescent analysis using the fourth primer pair, and FIG. 8(B) represents the results of gel electrophoresis using the fourth primer pair. Lane 1 corresponds to the negative control group. Lanes 2 to 7 correspond to 10⁶ to 10¹ CFU/mL of live MRSA samples. The PCR using the fourth primer pair can produce an amplification product having approximately 500 bp in length, in a sample with live MRSA bacteria. The results of either fluorescent analysis or gel electrophoresis indicate that the detection limit of the test kit of the present disclosure is determined to be 10³ CFU/mL live MRSA bacteria in a sample.

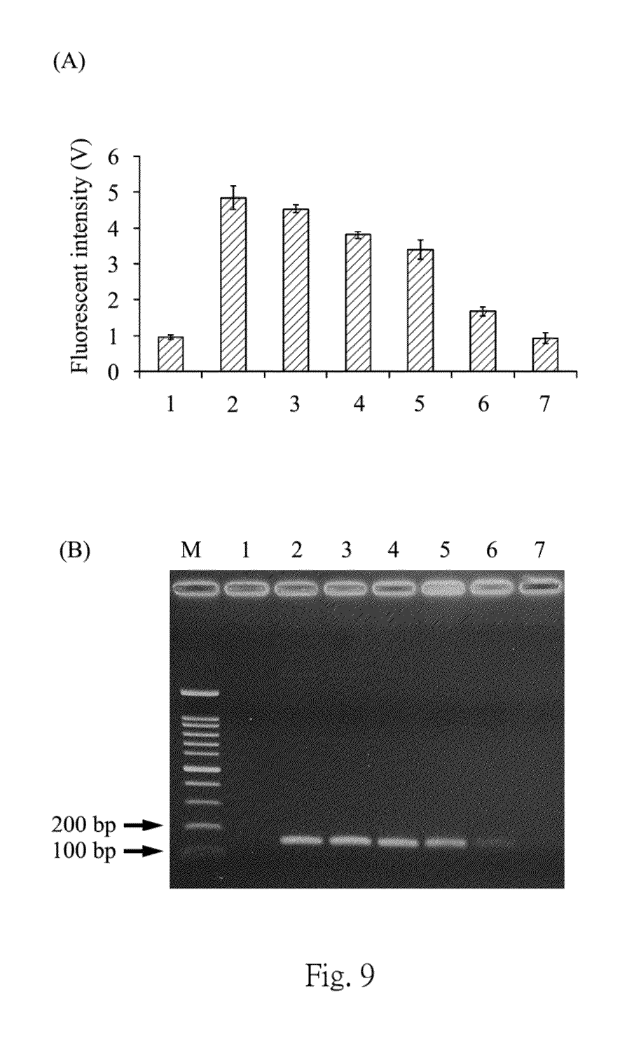

FIG. 9(A) represents the results of fluorescent analysis using the fifth primer pair, and FIG. 9(B) represents the results of gel electrophoresis using the fifth primer pair. Lane 1 corresponds to the negative control group. Lanes 2 to 7 correspond to 10⁶ to 10¹ CFU/mL of live Pseudomonas samples. The PCR using the fifth primer pair can produce an amplification product having approximately 153 bp in length. The results of either fluorescent analysis or gel electrophoresis indicate that the detection limit of the test kit of the present disclosure is determined to be 10² CFU/mL live Pseudomonas bacteria in the sample.

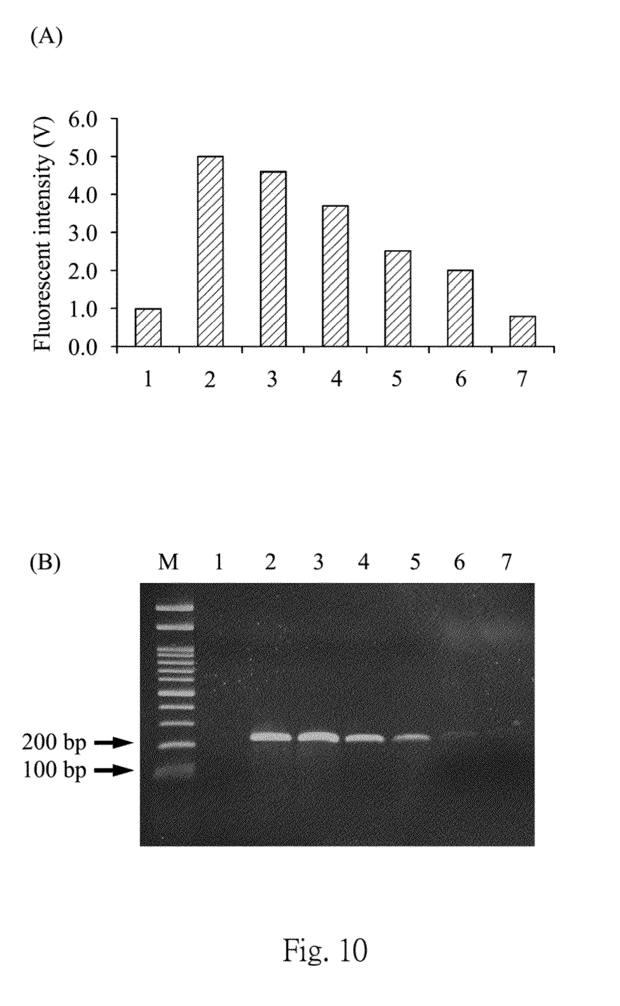

FIG. 10(A) represents the results of fluorescent analysis using the sixth primer pair, and FIG. 10(B) represents the results of gel electrophoresis using the sixth primer pair. Lane 1 corresponds to the negative control group. Lanes 2 to 7 correspond to 10⁶ to 10¹ CFU/mL of live E. coli samples. The PCR using the sixth primer pair can produce an amplification product having approximately 200 bp in length. The results of either fluorescent analysis or gel electrophoresis indicate that the detection limit of the test kit of the present disclosure is determined to be 10² CFU/mL live E. coli bacteria in a sample.

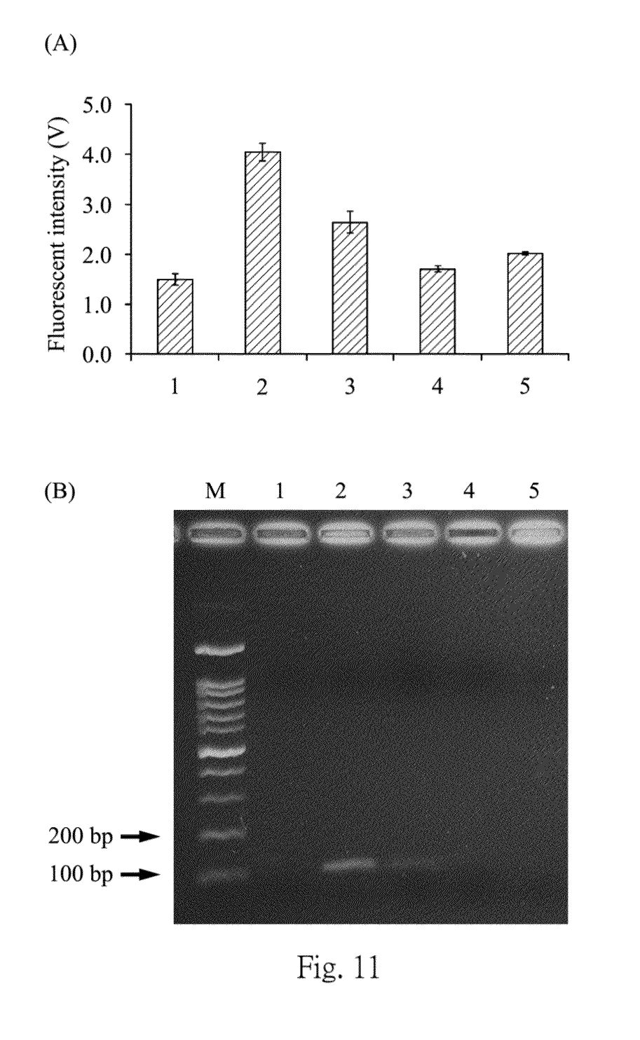

FIG. 11(A) represents the results of fluorescent analysis using the seventh primer pair, and FIG. 11(B) represents the results of gel electrophoresis using the seventh primer pair. Lane 1 corresponds to the negative control group. Lanes 2 to 5 correspond to 10⁴ to 10¹ CFU/mL of live Enterococcus samples. The PCR using the seventh primer pair can produce an amplification product having approximately 100 bp in length. The results of either fluorescent analysis or gel electrophoresis indicate that the detection limit of the test kit of the present disclosure is determined to be 10³ CFU/mL live Enterococcus bacteria in the sample.

According to the second embodiment of the present disclosure, the overall process of the method for rapidly determining an appropriate antibiotic, including sample purification, sample mixing, washing out of impurities, PCR and interpretation of reaction results, can be done only within 55 minutes by using the test kit of the present disclosure. Staphylococcus, CoNS and MRSA are three most common pathogens which cause nosocomial infections and which are difficult to treat, and antibiotics required for treatment of these pathogens are different. By using the test kit and the method thereof for rapidly determining an appropriate antibiotic of the present disclosure, clinicians are able to previously determine whether a patient carries a resistant bacterium before treatment, and thus can give proper medication for the disease to avoid delay in treatment.

Although the present disclosure has been described in considerable detail with reference to certain embodiments thereof, other embodiments are possible. Therefore, the spirit and scope of the appended claims should not be limited to the description of the embodiments contained herein.

It will be apparent to those skilled in the art that various modifications and variations can be made to the structure of the present disclosure without departing from the scope or spirit of the present disclosure. In view of the foregoing, it is intended that the present disclosure cover modifications and variations of the present disclosure provided they fall within the scope of the following claims.

What is claimed is:

1. A test kit for rapidly detecting a live bacterium, the test kit comprising:
ethidium monoazide adapted to intercalate into a DNA of a dead bacterium;
a magnetic bead bonded with vancomycin for capturing Gram-positive and Gram-negative bacteria;
a first primer pair comprising the nucleotide sequences referenced as SEQ ID NOs: 1 and 2; and
a microfluidic chip comprising:
a fluid storage unit comprising:
a first fluid storage chamber for storing a wash buffer; and
a second fluid storage chamber for storing a PCR reagent;
a reaction unit comprising a reaction chamber, a positive control reaction chamber and a negative control reaction chamber;
a fluid transportation unit comprising a pneumatic micro-pump and a transportation unit control air hole, wherein the fluid transportation unit is disposed between the fluid storage unit and the reaction unit for transporting the wash buffer or the PCR reagent to the reaction unit; and
a valve unit comprising:
a plurality of pneumatic micro-valves disposed between the fluid storage unit and the pneumatic micro-pump, and between the pneumatic micro-pump and the reaction unit; and
a plurality of valve control air holes for supplying air into the pneumatic micro-valves to control the opening and closing of the pneumatic micro-valves.

2. The test kit of claim 1, wherein the microfluidic chip further comprises a quantification pillar disposed on the pneumatic micro-pump.

3. A method for rapidly detecting a live bacterium, comprising:
providing the test kit of claim 1;
mixing the magnetic bead, the ethidium monoazide and a sample and exposing to visible light for 1 to 20 minutes, wherein the ethidium monoazide intercalates into the DNA of the dead bacterium, so that nucleic acid amplification of the dead bacterium by PCR is prevented;
collecting the magnetic bead;
loading the wash buffer in the first fluid storage chamber and activating the fluid transportation unit to pump the wash buffer into the reaction chamber via the pneumatic micro-valves for washing out unbound materials on the magnetic bead;
adding the first primer pair;
loading the PCR reagent in the second fluid storage chamber and activating the fluid transportation unit to pump the PCR reagent into the reaction chamber via the pneumatic micro-valves for performing PCR; and
detecting the presence of a PCR product, which is indicative of the existence of a live bacterium in the sample.

4. The method of claim 3, wherein the concentration of the ethidium monoazide is 0.01 to 30 mg/mL.

5. The method of claim 3, wherein the PCR product is detected by a gel electrophoresis system or an absorbance detection system.

6. The method of claim 4, wherein the PCR product is detected by a gel electrophoresis system or an absorbance detection system.

7. A test kit for rapidly determining an appropriate antibiotic, the test kit comprising:
ethidium monoazide adapted to intercalate into a DNA of a dead bacterium;
a magnetic bead bonded with vancomycin for capturing Gram-positive and Gram-negative bacteria;
a plurality of primer pairs comprising:
a first primer pair comprising the nucleotide sequences referenced as SEQ ID NOs: 1 and 2;
a second primer pair comprising the nucleotide sequences referenced as SEQ ID NOs: 3 and 4;
a third primer pair comprising the nucleotide sequences referenced as SEQ ID NOs: 5 and 6; and
a fourth primer pair comprising the nucleotide sequences referenced as SEQ ID NOs: 7 and 8; and
a microfluidic chip comprising a plurality of modules for simultaneously performing PCR, each of the modules comprising:
a fluid storage unit comprising:
a first fluid storage chamber for storing a wash buffer; and
a second fluid storage chamber for storing a PCR reagent;
a reaction unit comprising a reaction chamber, a positive control reaction chamber and a negative control reaction chamber;
a fluid transportation unit comprising a pneumatic micro-pump and a transportation unit control air hole, wherein the fluid transportation unit is disposed between the fluid storage unit and the reaction unit for transporting the wash buffer or the PCR reagent to the reaction unit; and
a valve unit comprising:
a plurality of pneumatic micro-valves disposed between the fluid storage unit and the pneumatic micro-pump, and between the pneumatic micro-pump and the reaction unit; and
a plurality of valve control air holes for supplying air into the pneumatic micro-valves to control the opening and closing of the pneumatic micro-valves.

8. The test kit of claim 7, further comprising a fifth primer pair comprising the nucleotide sequences referenced as SEQ ID NOs: 9 and 10, a sixth primer pair comprising the nucleotide sequences referenced as SEQ ID NOs: 11 and 12 and a seventh primer pair comprising the nucleotide sequences referenced as SEQ ID NOs: 13 and 14.

9. The test kit of claim 7, wherein the microfluidic chip further comprises a quantification pillar disposed on the pneumatic micro-pump.

10. A method for rapidly determining an appropriate antibiotic, comprising:
providing the test kit of claim 7;
mixing the magnetic bead, the ethidium monoazide and a sample and exposing to visible light for 1 to 20 minutes, wherein the ethidium monoazide intercalates into the DNA of the dead bacterium, so that nucleic acid amplification of the dead bacterium by PCR is prevented;
collecting the magnetic bead;
loading the wash buffer in the first fluid storage chamber and activating the fluid transportation unit to pump the wash buffer into the reaction chamber via the pneumatic micro-valves for washing out unbound materials on the magnetic bead;
adding the primer pair selected from the group consisting of the first primer pair, the second primer pair, the third primer pair and the fourth primer pair;
loading the PCR reagent in the second fluid storage chamber and activating the fluid transportation unit to pump the PCR reagent into the reaction chamber via the pneumatic micro-valves for performing PCR; and
determining an appropriate antibiotic according to an amplified PCR product detected.

11. The method of claim 10, wherein the primer pair further comprises a fifth primer pair comprising the nucleotide sequences referenced as SEQ ID NOs: 9 and 10, a sixth primer pair comprising the nucleotide sequences referenced as SEQ ID NOs: 11 and 12 and a seventh primer pair comprising the nucleotide sequences referenced as SEQ ID NOs: 13 and 14.

12. The method of claim 10, wherein the concentration of the ethidium monoazide is 0.01 to 30 mg/mL.

13. The method of claim 10, wherein the amplified PCR product is detected by a gel electrophoresis system or an absorbance detection system.

14. The method of claim 11, wherein the amplified PCR product is detected by a gel electrophoresis system or an absorbance detection system.

15. The method of claim 12, wherein the amplified PCR product is detected by a gel electrophoresis system or an absorbance detection system.
